(19) United States
(12) Patent Application Publication
(10) Pub. No.: US 2024/0009474 A1
(43) Pub. Date: Jan. 11, 2024

(54) DEVICE FOR MONITORING OPERATION OF A PROBE OF AN IMPLANTABLE ACTIVE CARDIAC DEVICE

(71) Applicant: SORIN CRM SAS, Clamart (FR)

(72) Inventors: Anne-Sophie Gandoin, Fresnes (FR); Nicolas Rosset, Chaville (FR); Sylvain Ploux, Gradignan (FR)

(21) Appl. No.: 18/029,866

(22) PCT Filed: Sep. 30, 2021
(86) PCT No.: PCT/EP2021/077032

§ 371 Date: Mar. 31, 2023

(30) Foreign Application Priority Data
Oct. 1, 2020 (FR) FR2010062

(51) Int. Cl. A61N 1/39

(52) U.S. Cl. CPC A61N 1/3943; A61N 1/3956

(57) ABSTRACT

A device for monitoring operation of a probe of an implantable active cardiac device, in particular an implantable automatic defibrillator or a defibrillator for cardiac resynchronization. The device comprising a parameter-determining device for determining values of a plurality of parameters characterizing the probe, and a processing unit configured to determine representative values that are representative of at least one parameter of the plurality of parameters characterizing the probe based on at least two different time scales. The processing unit is further configured to compare an analysis value of the at least one parameter of the plurality of parameters characterizing the probe with the representative values of the at least one parameter.

### TECHNICAL FIELD

The present invention relates to a device for monitoring operation of a probe of an implantable active cardiac device, in particular of an implantable automatic defibrillator or a defibrillator for cardiac resynchronization.

### TECHNICAL BACKGROUND

Probes are the critical part of an implantable active device system. Indeed, patients with implantable active devices, in particular implantable automatic defibrillators or defibrillators for cardiac resynchronization, are exposed to a significant risk of complications (up to 30%), the majority of which are related to inappropriate shocks. These shocks are often due to an alteration of the defibrillation probe, weakening the detection of the cardiac signal and thus taking into account noise for arrhythmia.

These failures may come about by an abrasion from friction between two probes (causing loss of insulation), by a rupture of the conductors, or even by a displacement bringing about poor contact between the probe tip and the heart wall.

### DESCRIPTION OF THE INVENTION

The task of the present invention is to improve the prediction of a failing probe.

The task of the present invention is achieved by means of a device for monitoring operation of a probe of an implantable active cardiac device, in particular an implantable automatic defibrillator or a defibrillator for cardiac resynchronization, comprising a parameter-determining device for determining values of a plurality of parameters characterizing the probe. The monitoring device includes a processing unit configured to determine values that are representative of at least one parameter of the plurality of parameters characterizing the probe based on at least two different time scales. The processing unit is further configured to compare a so-called analysis value of at least one parameter of the plurality of parameters characterizing the probe with the representative values of the said parameter.

By comparing the analysis value to the representative values according to two different time scales of at least one parameter characterizing the probe, the present device is configured to detect a failure of the probe.

Since the parameters to which the analysis value is compared are characteristic of the probe, a deviation between the representative values and the analysis value indicates a problem with the probe, and not, for example, a cardiac anomaly. In this way, the present device allows for an improvement in the prediction of a faulty probe.

The comparison, when considering two different time scales, allows for a further refinement of the reliability of detection of a failure of the probe.

The present invention, relating to a device for monitoring operation of a probe of an implantable active cardiac device, can be further improved by the following embodiments.

According to one embodiment, a first representative value may be an average of a first predefined number of representative values determined prior to the analysis value, which average is compared by means of the processing unit.

The said first representative value is therefore determined in such a way to represent a trend of a parameter characterizing the probe that is prior to the analysis value. The said first representative value may thus constitute a comparative value.

According to one embodiment, a second representative value may be an average of a second predefined number of representative values determined prior to the analysis value, which average is compared by means of the processing unit, wherein the said second predefined number is greater than the first predefined number.

In this way, two representative values can be determined on the basis of two different time scales.

The comparison of the analysis value while considering two different time scales further allows for a further refinement of the reliability of the detection of a failure of the probe. Indeed, the determination of a failure of the probe may depend on the time scale considered in relation to the analysis value.

According to one embodiment, a third representative value may be a rolling average based on an average of a third predetermined number of representative values determined prior to the analysis value, which average is compared by means of the processing unit, the said average of a third predetermined number of values corresponding to one parameter of the plurality of parameters. The rolling average is a type of statistical average that is particularly suitable for analyzing time series, in particular by suppressing transient fluctuations in such a way as to highlight longer-term trends.

Furthermore, the device is thus capable of determining three representative values based on three different time scales.

According to one embodiment, the processing unit can be configured during the determination of the representative values in such a way that one value, among the values of the plurality of parameters characterizing the probe that overruns a predefined limit value, is not taken into account.

In this way, it is possible to discard values that would not be considered usable or as being comprised within a viable range of values. Such values to be considered as being abnormal would then advantageously be discarded from the determination of the representative values in order to improve their reliability.

According to one embodiment, the processing unit may be configured to compare the analysis value of at least one parameter of the plurality of parameters characterizing the probe with the values that are representative of the said at least one parameter, the most recent value with respect to the analysis value that is taken into account for the determination of the representative values being within a first predetermined time interval.

It is thus possible to determine a first time interval which makes it possible to take into consideration only values for the determination of representative values starting out from an event which is not considered too old in relation to the analysis value.

In so doing, the reliability of the device, and therefore of the prediction of a faulty probe, is further improved.

According to one embodiment, the processing unit may be configured to compare the analysis value of at least one parameter of the plurality of parameters characterizing the probe with the values that are representative of the said at least one parameter, the oldest value with respect to the analysis value that is taken into account for determining the representative values being within a second predetermined time interval.

It is thus possible to determine a first time interval which makes it possible to take into consideration only values for the determination of representative values by going back in time to an event which is not considered too old in relation to the analysis value.

In so doing, the reliability of the device, and therefore of the prediction of a faulty probe, is further improved.

According to one embodiment, a parameter may be a parameter among an amplitude of the detection signal, a continuity of the probe, a daily detection percentage, a number of non-sustained ventricular fibrillations, a number of untreated ventricular fibrillations, a number of treated ventricular fibrillations, a number of isolated extrasystoles, a number of total extrasystoles, an impedance of the probe, and a pacing threshold. In this way the present device is configured to determine and to only take into account parameters characterizing a probe.

According to one embodiment, the plurality of parameters characterizing the probe may comprise at least two different parameters, in particular at least three different parameters.

Taking into account two different parameters, preferably three, characterizing the probe further improves the reliability and sensitivity of the present probe monitoring device.

According to one embodiment, the device may further comprise an alert unit for issuing an alert when the analysis value overruns in an increasing or decreasing manner a limit value of at least one representative value or/and a threshold limit of at least one parameter of the plurality of parameters.

The device is thus configured to issue an alert when a failure of the probe is determined when an analysis value is overrun. The term “threshold limit” of a parameter encompasses two aspects: both that of limit value (for example: the parameter is beyond a limit value) and that of limit variation (for example: the parameter varies by more than this limit variation).

According to one embodiment, each of the parameters of the plurality of parameters may respectively have one threshold limit, wherein the threshold limits are grouped into a first group of threshold limits for which the alert unit is configured to issue an alert in the event that a threshold limit of a single parameter is overrun, or a second group of threshold limits for which the alert unit is configured to issue an alert in the event that the threshold limits of at least two different parameters are overrun concomitantly.

In this way the present device is able to differentiate between the need or not to issue an alert as a function of the parameters the threshold limit of which is crossed. In this way only alerts considered as being justified are issued. As indicated above, the term “threshold limit” of a parameter includes two aspects: both that of limit value (for example: the parameter is beyond a limit value) and that of limit variation (for example: the parameter varies by more than this limit variation).

According to one embodiment, a threshold limit of a parameter assigned to the second group can be transferred to the first group if the overrunning of the said threshold limit occurs successively a predetermined number of times.

In this way, it is possible to adapt the sensitivity and specificity of the alerts during the monitoring of the probe can be adapted as a function of the overruns of the identified threshold limit.

According to one embodiment, threshold limits related to impedance of the probe, to continuity of the probe, and to the number of total extrasystoles may be part of the first group, and threshold limits related to the amplitude of a detection signal, to the detection percentage, to the pacing threshold, to the number of isolated extrasystoles, to the number of treated ventricular fibrillations, to the number of sustained but untreated ventricular fibrillations, and to the number of non-sustained ventricular fibrillations may be part of the second group.

In this way, the present device is adapted to discriminate threshold limits that relate to parameters that are sufficient on their own to warrant issuance of an alert.

According to one embodiment, a weighting value may be assigned to each parameter of the second group and wherein the alert unit may be configured to trigger an alert when the sum of the weighting values of the at least two parameters overruns a predetermined number.

The weighting of the parameters with respect to each other allows for a determination of whether their respective concomitant threshold limit overruns are sufficient to trigger the issuance of an alert.

According to one embodiment, the alert unit may comprise a memory unit configured to save a threshold limit overrun for a specified period of time and to delete it after the expiration of the said specified period of time.

The taking into consideration of previous events that have or were likely to trigger the issuance of an alert further improves the prediction of a faulty probe.

According to one embodiment, one parameter of the plurality of parameters may comprise a first threshold limit and a second threshold limit, wherein the first threshold limit is part of the first group and the second threshold limit is part of the second group.

Each of the threshold limits may correspond, for example, to threshold limits relating to different time scales. A first threshold limit may therefore relate to a discrete time value whereas the second threshold limit may relate to a variation of the parameter.

According to one embodiment, some of the threshold limits among the threshold limits of the second group may be linked to each other and others may be unlinked to each other, such that the alert unit may be configured to trigger an alert in the presence of at least two overruns of threshold limits among the thresholds of the second group that are not linked to each other.

The threshold limits of parameters can be linked to each other in the case in which they reflect the same problem (by way of example, the detection proportion and amplitude of detection). The overrun of two threshold limits that are linked to each other is thus not considered sufficient to issue an alert.

In this way, it may be necessary to have at least two overruns of threshold limits among the thresholds of the second group that are not linked to each other (such as, for example, the number of ventricular fibrillation episodes per day and the pacing threshold) to issue an alert.

### DESCRIPTION OF THE FIGURES

The invention and its advantages will be elucidated in more detail in the following by means of preferred embodiments and with particular reference to the following accompanying figures, wherein:

FIG. 1 represents a device for monitoring operation according to the present invention.

FIG. 2 represents the analysis of the variation of a parameter over a second time scale called “medium-term”.

FIG. 3 represents the analysis of the variation of a parameter over a third time scale called “long-term”.

FIG. 4a represents a first part of a flow chart relating to the analysis of variations of values of a parameter over three different time scales according to the present invention and to the raising of notices.

FIG. 4b represents a second portion of the flow chart illustrated in FIG. 4a.

FIG. 5 represents a flow chart relating to a triggering of an alert as a function of the notices raised according to the present invention.

FIG. 6 represents a table for weighting of the sufficiency of the notices among each other.

The invention will now be described in more detail using advantageous embodiments by way of examples and with reference to the figures. The described embodiments are merely possible configurations and it should be kept in mind that individual features as described above may be provided independently of each other or may be omitted altogether when implementing the present invention.

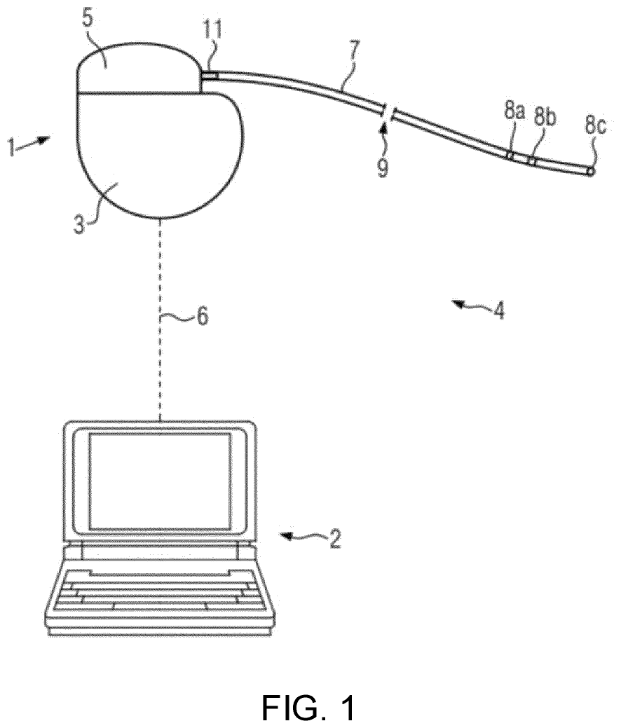

FIG. 1 illustrates an implantable active cardiac device 1 and a processing unit 2 forming a device 4 for monitoring operation of a probe according to the present invention.

The implantable active cardiac device 1 may be an implantable automatic defibrillator or a defibrillator adapted for cardiac resynchronization.

The implantable cardiac device 1 comprises a housing 3. The housing 3 comprises, in particular, electronic circuits and a battery, for example, of the lithium/iodine type. The housing 3 also comprises a connector part 5 into which an implantable probe 7 can be inserted and then screwed into place.

Although FIG. 1 shows an example of an implantable active cardiac device comprising an implantable probe 7, it should be kept in mind that in a variant (not shown), a plurality of implantable probes can be connected to the connector part 5 of the housing 3. The device 4 for monitoring operation of a probe is configured for an implantable active cardiac device comprising several probes. The device 4 for monitoring operation of a probe is thus configured to determine a failure resulting from abrasion from friction between two probes, causing at least partial loss of their insulation.

The implantable probe 7 comprises a plurality of electrodes 8a, 8b, 8c—wherein the number of electrodes illustrated in FIG. 1 is non-limiting—which constitute means for detection and pacing and/or defibrillation of the implantable cardiac device 1.

The implantable probe 7 may be a defibrillation probe.

The implantable probe 7 is configured to measure values of a plurality of parameters characterizing it, such as, for example, values of impedance.

According to the present invention, the plurality of parameters characterizing the implantable probe 7 may comprise at least: the amplitude of the detection signal, the continuity of the probe, a daily detection percentage, a number of non-sustained ventricular fibrillations, a number of untreated ventricular fibrillations, a number of treated ventricular fibrillations, a number of isolated extrasystoles, a number of total extrasystoles, an impedance of the probe, and a pacing threshold.

An isolated extrasystole is defined as a cardiac cycle with a single extrasystole.

In an embodiment in which the implantable probe 7 is a defibrillation probe, the following parameters may moreover also be considered: the continuity of the defibrillation probe, the number of treated ventricular fibrillations, the number of sustained but untreated ventricular fibrillations, and the number of non-sustained ventricular fibrillations.

A treated ventricular fibrillation is defined as a ventricular fibrillation that persists as such and which has been treated by electric shock.

An untreated ventricular fibrillation is defined as a ventricular fibrillation that persists as such but has not been treated by electric shock.

A non-sustained ventricular fibrillation is defined as a ventricular fibrillation that does not persist and has not been treated by electric shock.

It should be noted that although not all of the aforementioned parameters characterizing the probe are available for all types of probes, and as will be further explained in the following, this does not influence the multifactorial analysis implemented by the device 4 for monitoring operation of a probe of the present invention.

The implantable cardiac device 1 thus provides a parameter-determining device for determining values of a plurality of parameters characterizing the implantable probe 7.

The line breaks 9 indicate that the length of the implantable probe 7 is not fully shown in FIG. 1 not worries of drawing scale.

The implantable probe 7 is connected to the connector part 5 of the housing 3 by means of a male contact 11. A partial screwing or insufficient insertion of the male contact 11 into the connector part 5 of the implantable cardiac device 1 may cause connectivity problems.

As will be explained in more detail in the following, the device 4 for monitoring operation of a probe according to the present invention is configured to detect this type of failure.

To this end, the device 4 for monitoring operation of a probe according to the present invention moreover comprises a processing unit 2.

The processing unit 2 may be implemented in the implantable cardiac device 1 or in an external device, such as a computer.

The implantable cardiac device 1 and the processing unit 2 are configured to communicate with each other, for example not telemetry 6.

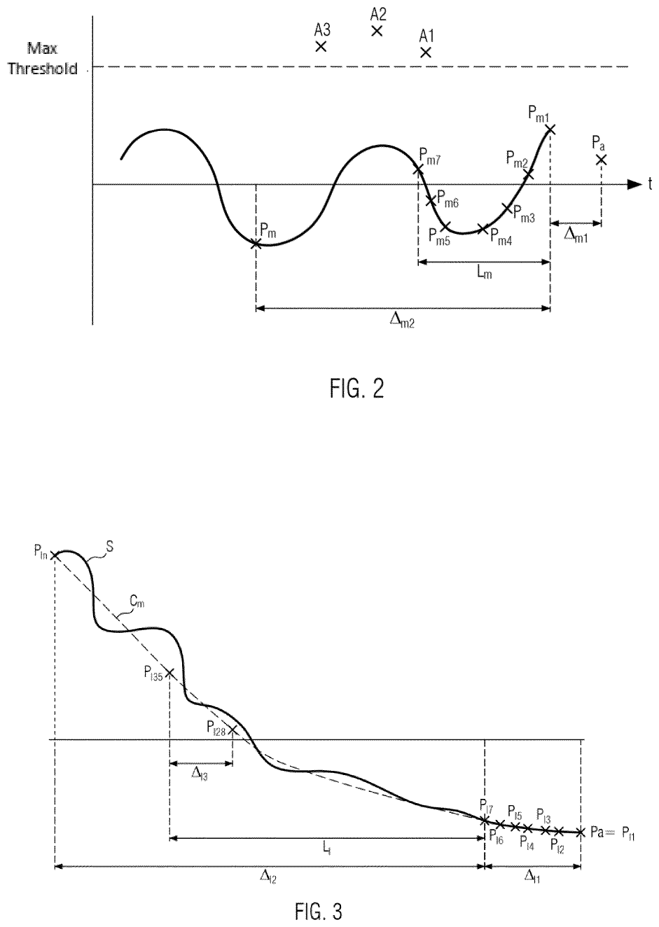

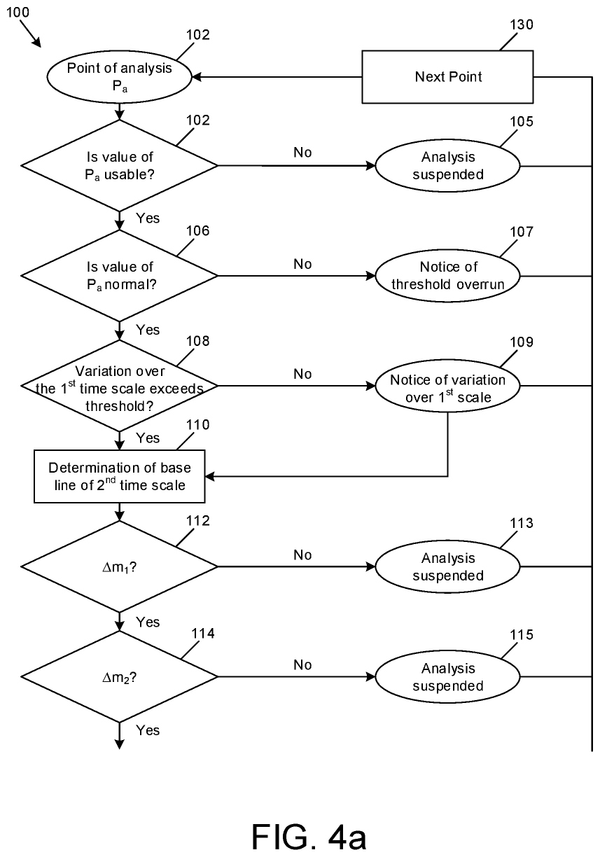

The processing unit 2 is configured to determine values that are representative of at least one parameter of the plurality of parameters characterizing the implantable probe 7 by basing on at least two different time scales, in particular three time scales. The analysis of variations in the values of a parameter over different time scales is further described with reference to FIG. 2 and FIG. 3.

The processing unit 2 is moreover configured to compare a so-called analysis value of at least one parameter of the plurality of parameters characterizing the implantable probe 7 with values that are representative of the said parameter.

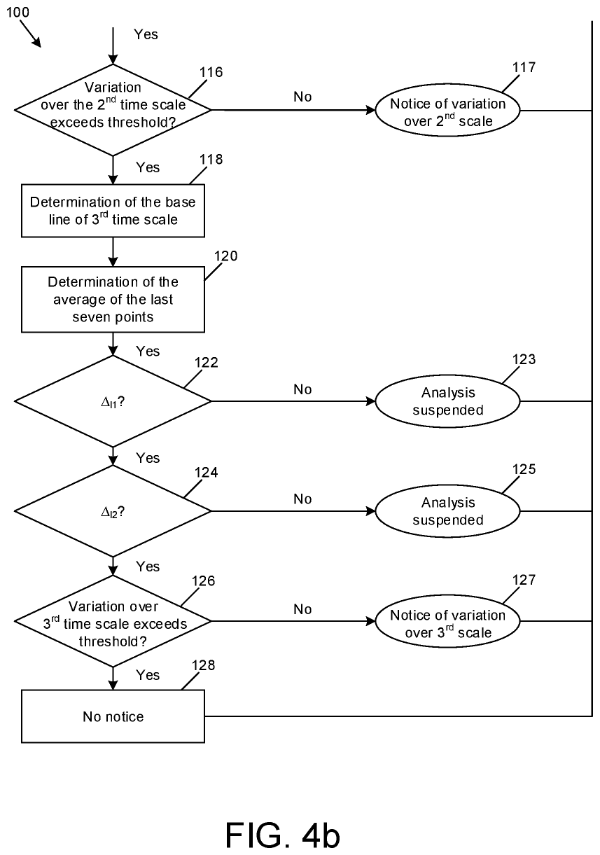

According to the present invention, the analysis of each of the parameters characterizing the implantable probe 7 may be performed according to several factors such as a maximum or minimum threshold, an absolute ascending or descending variation called “short-term” (for example, over one day), an absolute or relative ascending or descending variation called “medium-term” (for example, over one week) and a relative ascending or descending variation called “long-term” (for example over one month). The study of the variations of the parameters is described with reference to FIG. 2, FIG. 3, and FIG. 4.

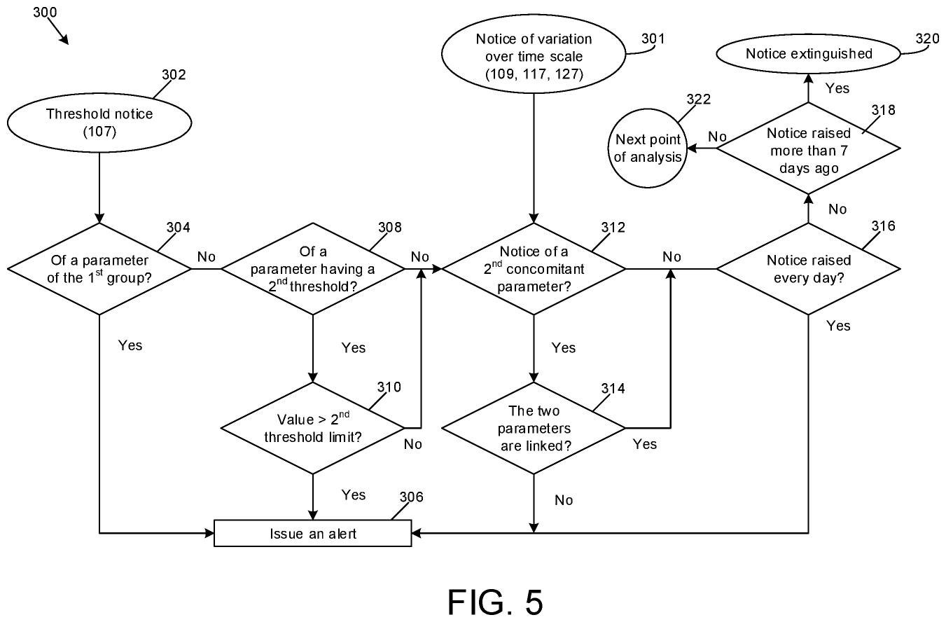

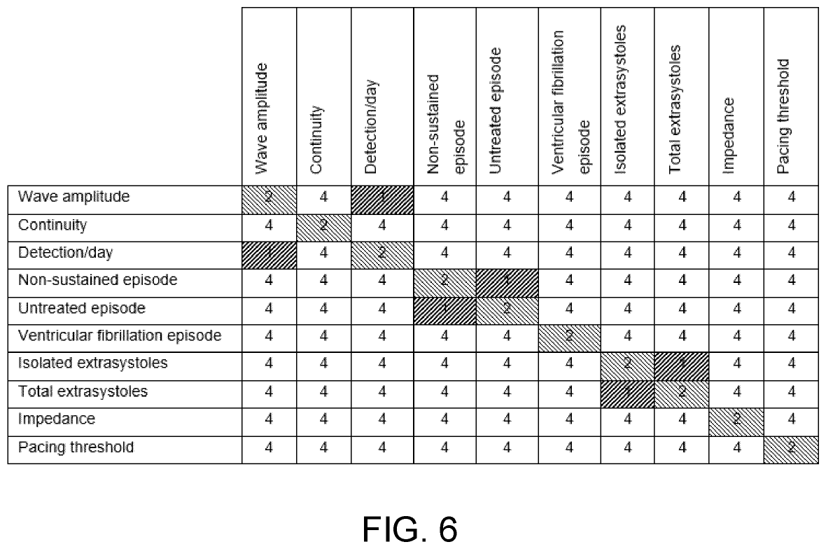

Secondly, a combination of all these analyses relating to the parameters characterizing the implantable probe is carried out in order to raise an alert, which is described with reference to FIG. 5 and FIG. 6.

It should be noted that the processing unit 2 is configured during the determination of the representative values in such a way that a value among the values of the plurality of parameters characterizing the probe that overruns a predefined limit value is not taken into account. A value overrunning such a predefined limit value is qualified to as a “non-usable point”, which is to say a point the value of which is outside a range of viable values or the value of which is not available. On the contrary, the value that overruns such a predefined limit value is qualified as an “abnormal point” when it is a point the value of which overruns a maximum or minimum threshold. The other values, which do not overrun a predefined limit value, are considered as “normal” and therefore usable for the analysis of the variation.

The analysis of the variation on a first time scale called “short-term” is done by analyzing the variations between a maximum point and a minimum point of the same day. By way of example, the processing unit 2 takes into account four impedance measurements during a day. In one variant, the processing unit 2 also takes into account other parameters such as the pacing threshold or the amplitude of detection. The measurement unit can take more or less than four measurements of a parameter over the course of a day.

The processing unit 2 further comprises an alert unit (not shown in FIG. 1). The alert unit is configured to issue an alert when the analysis value overruns in an increasing or decreasing manner a limit value of at least one representative value or/and of a threshold limit of at least one parameter of the plurality of parameters. The term “threshold limit” of a parameter includes two aspects: both that of a limit value (for example: the parameter is beyond a limit value) and that of a limit variation (for example: the parameter varies by more than this limit variation).

The processing unit 2 also comprises a memory unit (not shown in FIG. 1) by means of which data can be saved.

FIG. 2 shows the analysis of the variation according to a second time scale called “medium-term”.

The second time scale according to the present invention is different from the first time scale in that it refers to the analysis of variation over more than one day, in particular over one week.

The analyses of variations (relative or absolute) over the second so-called “medium-term” time scale are carried out between a point of analysis Pa and a baseline Lm called the medium-term baseline.

The point of analysis Pa corresponds to a point representative of the daily average.

The baseline Lm corresponds to a number n of last points each representative of the daily average. This baseline Lm includes only points qualified as “normal”, which is to say that points A1, A2, A3 in FIG. 2 are excluded from this baseline Lm inasmuch as they each have a value overrunning a maximum threshold (represented by a dotted horizontal line in FIG. 2). Points overrunning a minimum threshold can likewise be excluded from the baseline Lm.

Inasmuch as the baseline Lm is a “medium-term” baseline Lm, which is to say relative to one week, the medium-term baseline Lm corresponds to the last seven points Pm1 to Pm7 qualified as normal of the baseline Lm. As explained here above, each of the seven points Pm1 to Pm7 corresponds to a daily average.

In the example of FIG. 2, Pm1 represents the most recent point on the medium-term baseline Lm relative to the point of analysis Pa. Pm7 in turn represents the oldest point of the medium-term baseline Lm with respect to the point of analysis Pa.

In order to not compare the point of analysis Pa with events considered too old, a first time interval Δm1 can be determined between the point of analysis Pa and the most recent point Pm1 of the medium-term baseline Lm. This first time interval Δm1 may correspond to a duration of seven days. In the opposite case, the analysis of variations can be suspended.

Moreover, a second time interval Δm2 may be determined between the most recent point Pm1 of the medium-term baseline Lm and a point Pm of the baseline Lm that could correspond to the oldest point of the medium-term baseline Lm. This second time interval Δm2 may correspond to a duration of fourteen days. In the opposite case, the analysis of variations can be suspended.

FIG. 3 shows the analysis on the variation of values over a third time scale called “long-term”.

The processing unit 2 of the device 4 for monitoring operation of a probe according to the present invention considers a signal S obtained by means of the parameter determination device of the said monitoring device 4. The signal S may be a raw signal or a signal processed by known filtering means.

The processing unit 2 is configured to perform a rolling average, in particular over seven points, on the signal S. The curve Cm of FIG. 3 represents the averaged curve thus obtained. Averaging makes it possible to avoid alterations brought about by so-called “short-term” variations, which is to say variations over one day.

The curve Cm of FIG. 3 corresponds to the curve taken into consideration for the analysis of the variation over the third time scale known as long-term.

Since averaging creates a shift, the curve Cm can be refocused over three days in order to recalibrate the curve Cm.

The curve Cm shown in the example of FIG. 3 is a descending curve. In one embodiment, the curve Cm can be an ascending curve.

As for the analysis of the variation over the second time scale described in reference to FIG. 2, the points considered as being abnormal were excluded before the averaging operation. The points considered as being normal correspond to a weekly average.

As illustrated in FIG. 3, the predetermined time interval Δl1 may include the last seven points PI1 to PI7 which are considered to be normal, the first point PI1 corresponding to the point of analysis Pa, and the point PI7 corresponding to the oldest point with respect to the point of analysis Pa.

As shown in FIG. 3, in order to not compare the point of analysis Pa to events that are considered too old, the first time interval Δl1 is determined between the point of analysis Pa and the most recent point PI1 of the long-term baseline L1. This first time interval Δl1 may correspond to a duration of seven days. In the opposite case, the analysis of variations may be suspended.

In this way, by limiting the predetermined time interval Δl1 to the last seven points which are most recent with respect to the point of analysis Pa, it is not possible that there can be more than seven points between the point of analysis Pa and the last point PI7, which avoids the comparison of a weekly maximum or minimum with respect to events considered to be too old.

The points PI1 to PI7 of the predetermined time interval Δl1 are compared to a so-called long-term baseline L1.

In the example shown in FIG. 3, the so-called long-term baseline L1 corresponds to the last twenty-eight points considered to be normal of the curve Cm which points precede point PI7. The so-called long-term baseline L1 in the example of FIG. 3 is thus comprised between point PI7 and a point PI35.

In one variant of the present invention, the so-called long-term baseline L1 could comprise more or less than twenty-eight points, at least more points than the so-called medium-term baseline Lm.

In one variant, the so-called long-term baseline L1 may not comprise more than fifty-six points in order to avoid taking into consideration events considered as being too old.

The analysis of variation over the third time scale L1, called “long-term”, is done between the maximum, minimum or average of points considered to be normal over a predetermined time interval Δl1 and the long-term baseline L1 or the maximum, minimum or average of points that are considered to be normal over a predetermined time interval Δl3. The points of the curve Cm that are comprised in the predetermined time interval Δl1 are compared to points of the curve Cm comprised in a predetermined time interval Δl2 or Δl3.

The predetermined time interval Δl2 comprises the points between PI7 and Pln, with Pln corresponding to the oldest point relative to Pa. By way of illustration, Δl2 of FIG. 3 represents an interval comprising fifty-six points between PI7 and Pln.

The predetermined time interval Δl3 comprises the last seven points that are considered to be normal on the curve Cm starting from point PI35, which is the oldest point of the long-term baseline. In the example of FIG. 3, the predetermined time interval Δl3 thus comprises points PI28 through PI35.

When the curve Cm is descending as in the example of FIG. 3, the minimum of the points PI1 through PI7 of the predetermined time interval Δl1 can be compared to the maximum of the points PI28 through PI35 of the predetermined time interval Δl3.

In one variant, the average of points PI1 through PI7 of the predetermined time interval Δl1 may be compared to the average of the points PI28 through PI35 of the predetermined time interval Δl3.

FIG. 4a and FIG. 4b illustrate a flow chart 100 that is representative of the analysis of the variations and threshold values over three different time scales according to the present invention. The flow chart 100 is shown in two figures, FIG. 4a and FIG. 4b exclusively for clarity of the drawings. FIG. 4b illustrates the continuation of the steps shown in FIG. 4a. In this way, step 114 of FIG. 4a is followed by step 116 shown in FIG. 4b.

The flow chart 100 comprises steps implemented by the processing unit 2 of the device 4 for monitoring operation of a probe of an implantable active cardiac device 1 as described above. This therefore involves the analysis of variations in values of one parameter characterizing the implantable probe 7. As a consequence, the elements with the same numerical references already used for the description of FIG. 1 to FIG. 3 will not be described again in detail, and reference is made to their descriptions above.

In a first step 102 of the analysis of variations, the point of analysis Pa is taken into account by the processing unit 2.

In a step 104, it is determined whether the value of the point of analysis Pa corresponds to a usable value. If the value of the point of analysis Pa overruns a predefined limit value, it is qualified as a “non-usable point”, which is to say at a point the value of which is outside a range of viable values or the value of which is not available. In this case, the analysis is suspended at a step 105. A next point will then be considered for the point of analysis Pa at a step 130.

If the value of the point of analysis Pa is considered to be usable, the analysis continues.

In a step 106, it is determined whether the value of the point of analysis Pa corresponds to a value that can be considered a “normal value”. For this purpose, the value of the point of analysis is compared to a predefined limit, maximum or minimum threshold. If the value of the point of analysis Pa overruns the predefined maximum or minimum threshold, the value of the point of analysis Pa is considered to be abnormal. In this case, a notice indicating an overrun of a threshold is raised at a step 107 and a next point will then be considered for the point of analysis Pa.

If the value of the point of analysis Pa is considered to be normal, the analysis continues at a step 130.

In a step 108, it is determined whether the variation over the first time scale overruns a predetermined threshold limit, in the case in point, a limit variation. The first time scale may refer to a day. The variation over the first time scale then corresponds to the variation between a maximum point and a minimum point of a same day.

If the predetermined threshold for the first time scale is actually overrun, a notice indicating an overrun related to a variation is raised at a step 109.

Whether the predetermined threshold for the first time scale has been overrun or not in step 108, a baseline over a second time scale is determined in a step 110, wherein this second time scale is different from the first time scale. The second time scale may relate to a duration of one week.

In a step 112, it is determined whether the number of days between the point of analysis Pa and the most recent point Pm1 of the baseline Lm of the second so-called “medium-term” scale (see FIG. 2) is comprised within the predefined time interval Δm1. Preferably, Δm1 is equal to seven days.

If this is not the case, the analysis is suspended at a step 113. A next point will then be considered for the point of analysis Pa at a step 130.

Otherwise, the analysis continues.

At a step 114, it is determined whether the number of days between the most recent point Pm1 and the oldest point Pm of the baseline Lm of the second so-called “medium-term” scale (see FIG. 2) is comprised within the predefined time interval Δm2. Preferably, Δm2 is equal to fourteen days.

If this is not the case, the analysis is suspended at a step 115. A next point will then be considered for the point of analysis Pa at a step 130.

Otherwise, the analysis continues.

At a step 116, it is determined whether the variation over the second time scale overruns a predetermined threshold limit, in the case in point, a variation limit.

If the predetermined threshold for the second time scale is indeed overrun, a notice indicating an overrun with respect to a variation is raised at a step 117.

Whether the predetermined threshold for the second time scale has been overrun or not in step 116, a baseline over a third time scale is determined in a step 118, wherein this third time scale is different from the first time scale and the second time scale. The third time scale may relate to a month and is considered “long-term”.

In a step 120, an average of the last seven points of the so-called long-term baseline L1 (which is to say an average of the seven points starting from the oldest point of the so-called long-term baseline L1—see FIG. 3) is determined. In one variant, a maximum point or a minimum point of the last seven points of the so-called long-term baseline L1 may be determined at step 120.

In a step 122, it is determined whether the number of days between the point of analysis Pa corresponding to the most recent point PI1 and the oldest point Pln considered for analysis over the third time scale (see FIG. 3) is comprised within the predefined time interval Δl1. Preferably, Δl1 is equal to seven days.

If this is not the case, the analysis is suspended at a step 123. A next point will then be considered for the point of analysis Pa at a step 130.

Otherwise, the analysis continues.

At a step 124, it is determined whether the number of days between the most recent point PI1 and the oldest point PI of the baseline L1 of the so-called “long-term” third scale (see FIG. 3) is comprised within the predefined time interval Δl2. Preferably, Δl2 is equal to fifty-six days.

If this is not the case, the analysis is suspended at a step 125. A next point will then be considered as point of analysis Pa at a step 130.

Otherwise, the analysis continues.

At a step 126, it is determined whether the variation over the third time scale overruns a predetermined threshold limit, in the case in point, a variation limit.

If the predetermined threshold for the third time scale is indeed overrun, a notice indicating an overrun with respect to a variation is raised at a step 127.

Otherwise, no notice is raised at a step 128.

In the two cases, whether the predetermined threshold has been overrun or not, the analysis continues by taking into consideration a next point for the point of analysis Pa at a step 130.

The analysis of variations illustrated by flow chart 100 therefore comprises successive variation analyses over different time scales, from the shortest time scale to the longest time scale.

FIG. 5 depicts a flow chart 300 relating to a triggering of an alert as a function of the notices raised in steps 107, 109, 117 and 127 of flow chart 100.

The flow chart 300 comprises steps implemented by the processing unit 2 of the device 4 for monitoring operation of a probe of an implantable active cardiac device 1 as described above. As a consequence, the elements with the same numerical references already used for the description of FIG. 1 to FIG. 4 will not be described again in detail, and reference is made to their descriptions above.

The flow chart 300 illustrates how the various notices that have previously been raised in steps 107, 109, 117 and 127 of flow chart 100 are combined in order to optimize the sensitivity and specificity of the alerts sent to the physician. In other words, in order to exclusively trigger justified alerts.

According to the present invention, a notice is different from an alert. The alert is communicated to the physician from the start to indicate a potential failure of the probe, for example, by means of a visual or audible message. A notice is not necessarily communicated to the physician. As will be explained in the following, the concomitant occurrence of notices can however lead to the triggering of an alert.

In this way, whereas an analysis of values that are above or below a threshold limit can immediately raise an alert (for example, in the case of an impedance or a continuity), an analysis of variation of this same parameter must go through a notice step.

Two types of notices can be taken into account by the processing unit 2 of the monitoring device 4 of the present invention: notices of variations (at a step 301) and notices of threshold (at a step 302).

As described with reference to step 107 of FIG. 4a, a notice of a threshold corresponds to the crossing of a threshold limit by the value of the point of analysis Pa.

As described with reference to steps 109 of FIG. 4a and steps 117 and 127 of FIG. 4b, a notice of variations corresponds to the crossing of a threshold limit by the variation according to a time scale of the value of a parameter characterizing the implantable probe 7.

At least one threshold limit is determined for each parameter characterizing the implantable probe 7.

A plurality of threshold limits can be determined for the same parameter. In this way, a parameter may have a first threshold limit, the overrunning of which generates a notice, and a second threshold limit, the overrunning of which generates an alert.

According to the present invention, the threshold limits of the parameters characterizing the probe can be classified into two groups.

The first group brings together the threshold limits for which the alert unit of the monitoring device 4 is configured to issue an alert in the case of an overrun of a threshold limit of a single parameter. By way of example, the thresholds relating to impedance of the probe, to the continuity of the probe, and to the number of total extrasystoles belong to the first group.

The second group brings together the threshold limits for which the alert unit of the monitoring device 4 is configured to issue an alert in the case of a concomitant overrun of the threshold limits of at least two different parameters. By way of example, the threshold limits relating to the amplitude of a detection signal, to the detection percentage, to the pacing threshold, to the number of isolated extrasystoles, to the number of treated ventricular fibrillations, to the number of sustained but untreated ventricular fibrillations, and to the number of non-sustained ventricular fibrillations belong to the second group.

It should be noted that a threshold limit of a parameter assigned to the second group can be transferred to the first group if the overrunning of the said threshold limit occurs successively a predetermined number of times.

It should also be noted that the threshold limit, in and of itself, for raising an alert may vary. This is the case, for example, for the impedance of a left ventricular probe: a notice can be raised for a unipolar vector on a threshold limit that is lower than for a bipolar vector.

As illustrated in FIG. 5, if it is detected at a step 302 of flow chart 300 that a threshold notice has been raised (at step 107 of flow chart 100), it is determined at a step 304 whether the raised threshold notice relates to a value of a parameter classified in the first group.

If this were to be the case, this condition is sufficient for an alert to be issued by the alert unit of the monitoring device 4 at a step 306. As explained earlier with reference to the threshold limits of the first group, an analysis of values overrunning a threshold limit can indeed allow the immediate raising of an alert (for example, in the case of an impedance or a continuity). In this way, a very high or very low value of a parameter (for example, an impedance of the probe greater than 2000 ohms) is, in and of itself, a characteristic of a probe problem (in favor of a fracture). This factor can therefore be sufficient in itself to trigger an alert indicating a potential probe failure.

Otherwise, it is checked in a step 308 whether the raised threshold notice relates to a parameter comprising a second threshold limit, the overrunning of which is likely to trigger an alert. Indeed, as explained above, some parameters, for total extrasystoles, for example, may have a first threshold limit the overrunning of which generates a notice, and a second threshold limit the overrunning of which generates an alert. In this case, it is checked at a step 310 whether the value of the point of analysis crosses the second threshold limit. If so, an alert is triggered in step 306.

The cases that are not covered above by the notices that can generate an alert on their own are described in the following.

As illustrated by the flow chart 300 at step 301, a notice of variations of one parameter is not sufficient on its own to raise an alert.

This parameter therefore needs to have at least a second concomitant parameter for an alert to be triggered.

It is thus determined at a step 312 whether a notice relating to a second parameter has been detected in a concomitant manner with the notice of step 301. This second parameter may also not be sufficient in the case that it reflects the same problem (by way of example, the detection proportion and the amplitude of detection). It is then said that the first and second parameters are “linked”.

This is why, in a step 314, it is determined whether the first parameter and the second parameter are linked to each other.

If they are not linked to each other, then the concomitance of a notice relating to a first parameter with a notice relating to a second parameter, which second parameter is not linked to the first parameter, brings about the triggering of an alert at step 306.

It is therefore necessary to have at least one second unlinked parameter, such as, for example, the number of episodes of ventricular fibrillation per day or the pacing threshold, to raise an alert.

The pairs of parameters characterizing the probe that are not sufficient for one another to trigger an alert because they are “linked” are illustrated by means of the shaded boxes in FIG. 6, which will be further described below.

Three pairs of “linked” parameters are thus defined. The first pair corresponds to the detection/day and to the wave amplitude. The second pair corresponds to a sustained episode and to an untreated episode. Lastly, the third pair corresponds to isolated extrasystoles and to total extrasystoles.

If it is determined in step 314 of flow chart 300 that the two parameters are linked, or even that in step 312 a second parameter notice had not been detected in a concomitant manner, it is determined in a step 316 whether the notice relating to the first parameter (the one in step 301) is triggered each day.

For this purpose, the notices related to the first parameter are saved in a memory unit of the processing unit 2.

It should be noted that the analysis of the different parameters is done simultaneously. When one of the parameters raises a notice, the notice remains active for a predetermined period, for example, seven days.

If the notice is raised several days in a row, then it will remain active for the seven days following the end of its raising.

If a plurality of notices of different parameters are active at the same time (which is to say in a concomitant manner), this may activate an alert.

This system of concomitance of the alerts makes it possible to detect a failure that can occur in different ways at different times.

In a step 318, it is determined whether the notice was triggered more than seven days ago. If this is the case, the said notice is deactivated (extinguished) in step 320. Otherwise, the analysis continues at step 322, taking into consideration the next point.

FIG. 6 represents a weighting table for the sufficiency of notices among each other.

In order to calculate the sufficiency of the notices among each other, in particular in step 314 of the flow chart 300, a pair-by-pair weighting scheme is implemented.

All notices related to the parameters characterizing the probe that appear in a day are classified in alphabetical order.

The hatched boxes in FIG. 6 represent one single notice, not two notices raised from the same parameter, such as the variation over two different scales of a same parameter.

The “weightings” assigned to each pair in the table are added together. If the result of the said addition is greater than and different from 3, the alert is raised at step 306 of the flow chart 300 (see FIG. 5).

It is then necessary to go to the line of the first parameter, and then add the weighting of each of the pairs formed to the respective weighting of the first parameter (indicated in the hatched boxes). Examples are given below.

In a first example, the first parameter corresponds to the impedance and the second parameter corresponds to the pacing threshold. For the first example, we must first go to the impedance line and add the respective weighting of the impedance (which is to say 2, see the hatched box) and the weighting of the pair formed with the pacing threshold (which is to say 4). The result of the addition, which is 6, is greater than 3: an alert is therefore raised.

In a second example, the first parameter corresponds to the amplitude of the signal and the second parameter corresponds to the daily proportion of signal detected, which is to say the daily proportion of signal in spontaneous rhythm. For the second example, it is therefore necessary to go to the wave amplitude line and add the respective weighting of the wave amplitude (which is to say 2, see the hatched box) and the weighting of the pair formed with the detection/day (which is to say 1). The result of the addition being equal to 3, the alert is not raised.

In one embodiment of the invention, the device for monitoring operation of a probe takes into consideration at least two different parameters characterizing the probe.

In another embodiment of the invention, the device for monitoring operation of a probe takes into consideration at least three different parameters characterizing the probe. In this way, a third example is described below in which three different parameters are taken into account.

In the third example, the first parameter corresponds to the wave amplitude, the second parameter corresponds to the detection/day and the third parameter corresponds to the continuity. For the third example, it is therefore necessary to go to the line of the wave amplitude, and to add the respective weighting of the wave amplitude (which is to say 2, see the hatched box), the weighting of the pair formed with the detection/day (which is to say 1) and the weighting of the pair formed with the continuity (which is to say 4). The result of the addition being equal to 7, which is to say greater than 3, the alert is raised.

The present invention thus allows for the taking into consideration of multiple parameters (electrical and rhythmic) that are characteristic of an implantable probe over different time scales in order to improve the prediction of a failure of the implantable probe.

1-17. (canceled)

18. A device for monitoring operation of a probe of an implantable active cardiac device, in particular an implantable automatic defibrillator or a defibrillator for cardiac resynchronization, comprising:
a parameter-determining device for determining values of a plurality of parameters characterizing the probe, and
a processing unit configured to determine representative values that are representative of at least one parameter of the plurality of parameters characterizing the probe based on at least two different time scales,
wherein the processing unit is further configured to compare an analysis value of the at least one parameter of the plurality of parameters characterizing the probe with the representative values of the at least one parameter.

19. The device for monitoring operation of a probe of an implantable active cardiac device according to claim 18, wherein a first representative value is an average of a first predefined number of the representative values determined prior to the analysis value, wherein the analysis value and the average of the first predefined number of the representative values are compared by the processing unit.

20. The device for monitoring operation of a probe of an implantable active cardiac device according to claim 19, wherein a second representative value is an average of a second predefined number of the representative values determined prior to the analysis value, wherein the analysis value and the average of the second predefined number of representative values are compared by the processing unit, and wherein the second predefined number is greater than the first predefined number.

21. The device for monitoring operation of a probe of an implantable active cardiac device according to at least one of claim 18, 19, or 20, wherein a third representative value is a rolling average based on an average of a third predetermined number of the representative values determined prior to the analysis value, wherein the analysis value and the rolling average of the third predetermined number of the representative values are compared by the processing unit, and wherein the rolling average and the third predetermined number of representative values corresponds to one of the plurality of parameters.

22. The device for monitoring operation of a probe of an implantable active cardiac device according to claim 18, wherein the processing unit is configured during the determination of the representative values in such a way that one value, among the values of the plurality of parameters characterizing the probe that overruns a predefined limit value, is not taken into account.

23. The device for monitoring operation of a probe of an implantable active cardiac device according to claim 18, wherein the processing unit is configured to compare the analysis value of the at least one parameter of the plurality of parameters characterizing the probe with the values that are representative of the at least one parameter, wherein a most recent value, with respect to the analysis value that is taken into account for the determination of the representative values, is within a first predetermined time interval.

24. The device for monitoring operation of a probe of an implantable active cardiac device according to claim 23, wherein the processing unit is configured to compare the analysis value of the at least one parameter of the plurality of parameters characterizing the probe with the values that are representative of the at least one parameter, wherein the most recent value, with respect to the analysis value that is taken into account for the determination of the representative values, is within a second predetermined time interval.

25. The device for monitoring operation of a probe of an implantable active cardiac device according to claim 18, wherein the at least one parameter is one of an amplitude of a detection signal, a continuity of the probe, a daily detection percentage, a number of non-sustained ventricular fibrillations, a number of untreated ventricular fibrillations, a number of treated ventricular fibrillations, a number of isolated extrasystoles, a number of total extrasystoles, an impedance of the probe, and a pacing threshold.

26. The device for monitoring operation of a probe of an implantable active cardiac device according to claim 18, wherein the plurality of parameters characterizing the probe comprises at least two different parameters.

27. The device for monitoring operation of a probe of an implantable active cardiac device according to claim 18, further comprising an alert unit for issuing an alert when the analysis value overruns in an increasing or decreasing manner a limit value of at least one representative value or/and a threshold limit of at least one parameter of the plurality of parameters.

28. The device for monitoring operation of a probe of an implantable active cardiac device according to claim 27, wherein each parameter of the plurality of parameters respectively has a threshold limit, wherein the threshold limits are grouped into:
a first group of threshold limits for which the alert unit is configured to issue an alert in the event that a threshold limit of a parameter is overrun, or
a second group of threshold limits for which the alert unit is configured to issue an alert in the event that threshold limits of at least two parameters are overrun concomitantly.

29. The device for monitoring operation of a probe of an implantable active cardiac device according to claim 28, wherein a threshold limit of a parameter assigned to the second group is transferred to the first group if an overrunning of the threshold limit occurs a predetermined number of times, successively.

30. The device for monitoring operation of a probe of an implantable active cardiac device according to claim 28, wherein:
the first group of threshold limits relate to at least one of an impedance of the probe, a continuity of the probe, and a number of total extrasystoles, and
the second group of threshold limits relate to an amplitude of a detection signal, a detection percentage, a pacing threshold, a number of isolated extrasystoles, a number of treated ventricular fibrillations, a number of sustained but untreated ventricular fibrillations, and a number of non-sustained ventricular fibrillations.

31. The device for monitoring operation of a probe of an implantable active cardiac device according to claim 28, wherein a weighting value is assigned to each parameter of the second group and wherein the alert unit is configured to trigger an alert when a sum of the weighting values of the at least two parameters overruns a predetermined number.

32. The device for monitoring operation of a probe of an implantable active cardiac device according to claim 28, wherein the alert unit comprises a memory unit configured to save a threshold limit overrun for a specified period of time and wherein the memory unit is configured to delete the threshold limit after expiration of the specified period of time.

33. The device for monitoring operation of a probe of an implantable active cardiac device according to claim 28, wherein a parameter of the plurality of parameters comprises a first threshold limit and a second threshold limit, wherein the first threshold limit is part of the first group and the second threshold limit is part of the second group.

34. The device for monitoring operation of a probe of an implantable active cardiac device according to claim 28, wherein a first set of the threshold limits among the threshold limits of the second group are linked to each other and a second set of threshold limits among the threshold limits of the second group are not linked to each other, wherein the alert unit is configured to trigger an alert in response to at least two overruns of threshold limits among the second set of threshold limits.
